(19) United States
(12) Patent Application Publication
(10) Pub. No.: US 2014/0358228 A1
(43) Pub. Date: Dec. 4, 2014

(54) TEMPORARY PERIODONTAL PROSTHETIC DEVICE THAT GIVES THE APPEARANCE OF FULLER LIPS

(71) Applicant: Tyna MAREE DEGENHARDT, Huntington Beach, CA (US)

(72) Inventor: Tyna MAREE DEGENHARDT, Huntington Beach, CA (US)

(21) Appl. No.: 13/960,542

(22) Filed: Aug. 6, 2013

Related U.S. Application Data
(60) Provisional application No. 61/828,014, filed on May 28, 2013.

(51) Int. Cl. A61F 2/00

(52) U.S. Cl. CPC A61F 2/0059
USPC 623/11.11

(57) ABSTRACT

A temporary periodontal prosthetic device that gives the appearance of fuller lips. The prosthetic is a flexible strip which may be placed on the upper or lower gum and attached to the gum with a gentle adhesive backing. The fullness of the wearer's lips is consequently enhanced by the prosthetic pushing the lips outwards. The prosthetic is attachable and removable without the discomfort, cost, risks, or permanence of a surgical procedure. The prosthetic may be adjustable to fit a wearer's gums. The adhesive backing may be protected by a removable release liner prior to use. The prosthetic may also be configured to have a flavor or freshen the breath.

### CROSS-REFERENCE TO RELATED APPLICATIONS

This application claims priority to U.S. Provisional Patent Application No. 61/828,014, filed on Jun. 5, 2013, which is hereby incorporated by reference in its entirety. Priority is claimed pursuant to 35 U.S.C. §119.

### STATEMENT RE: FEDERALLY SPONSORED RESEARCH/DEVELOPMENT

Not applicable

### BACKGROUND

1. Technical Field

The present disclosure relates generally to cosmetic lip augmentation. More particularly, the present disclosure relates to the use of an easily applicable and removable prosthetic for cosmetic lip augmentation.

2. Related Art

For many, the fullness of a person's lips is associated with youth and facial beauty. Lips are a prominent facial feature, and for many, may represent a source of unhappiness due to their lips' appearance not naturally having a desired fullness.

To that extent, a large variety of methods have been developed and practiced in order to artificially augment lip fullness. However, most existing and currently practiced methods of lip augmentation require cosmetic surgery. Such methods typically involve injections or implantations of foreign or transplanted material into a person's lips. Common injected substances include collagens, lipids, or polysaccharides such as hyaluronic acid, and common implanted substances include allografts, autografts, and expanded polytetrafluoroethylene. Unfortunately, these methods suffer from various deficiencies, including the high costs of surgery, risks of the immune system rejecting foreign materials, pain and discomfort, and high barriers to reversibility.

Consequently, there is a need for an improved apparatus and method for cosmetic lip augmentation.

### BRIEF SUMMARY

To solve these and other problems, a temporary periodontal prosthetic device for cosmetic lip augmentation is contemplated wherein the wearer may adhesively attach a space filling element to the upper or lower gums, or gingiva, increasing the apparent fullness of the wearer's lips. A complete augmentation may be achieved by wearing a space filling element on each of the upper and lower gums.

The removable prosthetic may be a space filling element with an inner face and an outer face. The space filling element may have a size and configuration allowing it to be placed in a person's mouth, fitting comfortably against the upper and lower gums. When placed as such, it is preferable that the space filling element not protrude above the gingival margin of the lower gum, or below the gingival margin of the upper gums, as it may then become visible to onlookers when a wearer's teeth are exposed, such as when the wearer is smiling.

An adhesive may be placed on the inner face of the space filling element. The adhesive may be strongly adhered to the space filling element, but may also adhere only temporarily to the upper or lower gums, so that the wearer may remove or adjust the location of the prosthetic without great difficulty or pain.

It may thus be seen that when the prosthetic is placed against the gum, the lips may be pushed outward by the outer face of the space filling element, resulting in the wearer having the appearance of fuller lips.

It may be preferable that the space filling element be formed of a gel-type plastic. Suitable materials may include polyethelene, polyurethane, polypropylene, polystyrene, polyvinyl chloride, polytetrafluoroethylene, expanded polytetrafluoroethylene or silicone. It may be more preferable that silicone gel be used to form the space filling element.

The adhesive may be, for example, acrylate, methylacrylate, diacrylate, cyanoacrylate, abietic acid, poly(methylvinylether-co-maleic acid) or polyvinyl acetate. It may be more preferable that poly(methylvinylether-co-maleic acid) be used. The adhesive may also be a pressure-sensitive adhesive or a moisture-sensitive adhesive.

The prosthetic may also have a removable release liner attached to the adhesive. Such a release liner may be removed by the wearer just prior to placement of the prosthetic.

The space-filling element may have one or more marked segment lines. These segment lines may be utilized for modifying the size of the prosthetic to better fit the size of the wearer's upper or lower gums. The segment lines, for example, may be evenly spaced marks which may serve as guides for cutting. The segment lines may also have one or more slits or perforations so as to allow the size modification to be performed by tearing, making scissors or other cutting devices unnecessary.

The prosthetic may also be flavored or serve to freshen the breath. For example, the space-filling element may have a flavored or breath-freshening coating, or be impregnated with a flavored or breath-freshening substance.

The prosthetic may be worn by the wearer placing it in the preferred location and configuration, and adhering the adhesive to the upper or lower gum. A release liner may have to be removed prior to placement, so as to expose the adhesive. The wearer may also resize the prosthetic to fit their upper or lower gum, preferably by cutting or tearing along a provided marked segment line.

### BRIEF DESCRIPTION OF THE DRAWINGS

These and other features and advantages of the various embodiments disclosed herein will be better understood with respect to the following description and drawings, in which:

FIG. 1 is an upper perspective view of an embodiment of the present invention, showing a space filling element with removable segments and a removable release liner;

FIG. 2 is an upper perspective view of the space filling element showing removal of segments from the space filling element for selectively sizing the space filling element;

FIG. 3 is a cross-sectional view taken upon line 3 of FIG. 3, showing a cross section along a transverse plane of an embodiment of the present invention;

FIG. 4 is a cross-sectional view of an alternative embodiment of the present invention;

FIG. 5 is a cross-sectional view of another alternative embodiment of the present invention;

FIG. 6 is a profile illustration of a human face shown in phantom, and human gums and human teeth defining an oral cavity; and

FIG. 7 is a profile illustration of profile illustration of a human face shown in phantom, human gums and human teeth defining an oral cavity, and an embodiment of the present invention being worn.

Common reference numerals are used throughout the drawings and the detailed description to indicate the same elements.

### DETAILED DESCRIPTION

As described in more detail below, various aspects of the present invention related to a temporary periodontal prosthetic device for cosmetic lip augmentation which has a space filling element with an inner surface and an outer surface. An adhesive may be attached to the inner surface of the space filling element for selectively adhering to an upper or lower gum, or gingiva of a wearer. The adhesive may hold the space filling element in place adjacent to the upper or lower gum, and the prosthetic device may be removable without great difficulty or pain. When the prosthetic device is disposed in the human mouth adjacent to an upper or lower gingiva, the outer surface of the prosthetic device may push a lip outward, resulting in the wearer's appearance being enhanced through an increase in perceived lip fullness. The fullness of one or both lips may be augmented in such a fashion. Such an enhanced appearance may be similar to the appearance obtainable by surgical augmentation of lips, but without the high costs of surgery, the risks of the immune system rejecting foreign materials, the associated pain and discomfort, and the high barrier to reversibility.

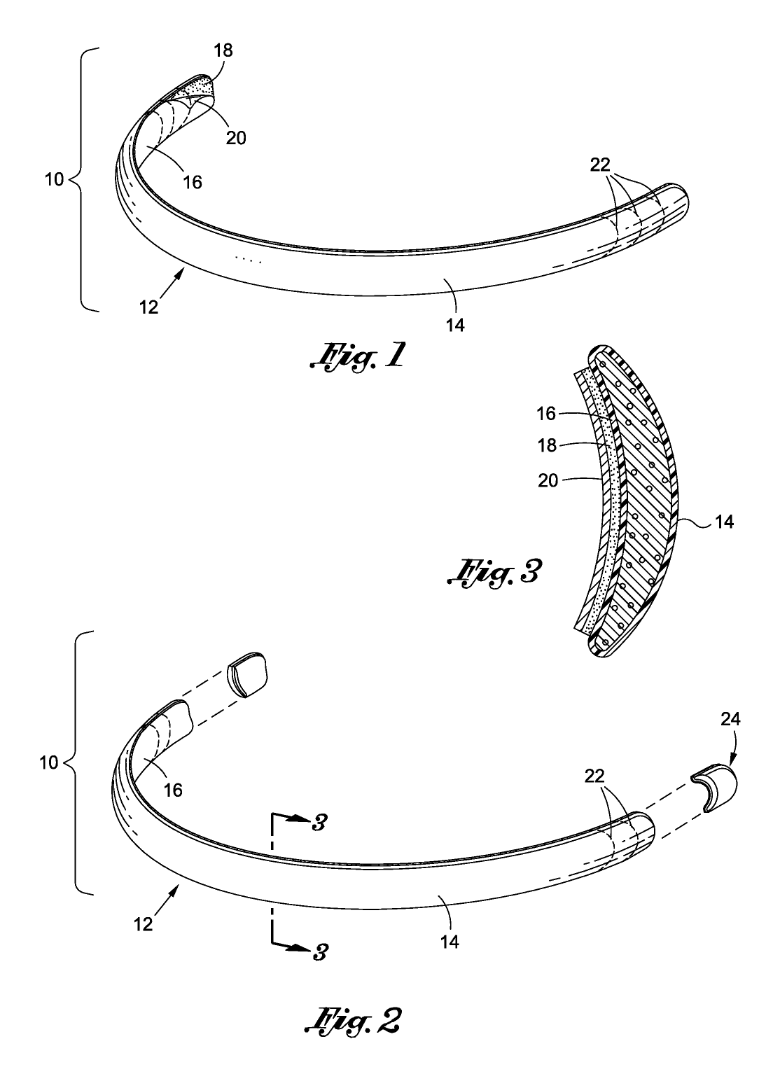

Referring now to the drawings, and more specifically to FIG. 1, a removable prosthetic device 10 according to an embodiment of the present invention is shown. The prosthetic device 10 may comprise a space filling element 12 having an outer face 14 and an inner face 16. On the inner face 16 may be disposed an adhesive 18. The adhesive 18 may have disposed over it a release liner 20. It may be preferable that the prosthetic device 10 be formed or formable into an arcuate configuration as illustrated for placement within the human oral cavity against the gums, or gingiva. In one embodiment, the space filling element 12 of the prosthetic device 10 may be more rigid and may be produced in a predefined arcuate configuration. In another embodiment, the space filling element 12 of the prosthetic device 10 may be formed of a generally planar strip of a flexible material, and may be selectively transitioned between a generally planar configuration and a generally arcuate configuration by the wearer, wherein the generally arcuate configuration may be so configured to fit the wearer's upper or lower gum.

In order to provide such flexibility, with an additional benefit of providing wearer comfort, it may be preferable that the space filling element 12 of the prosthetic device 10 be formed of a gel-type plastic. Such plastics may include, for example but without limitation, polyethelene, polyurethane, polypropylene, polystyrene, polyvinyl chloride, polytetrafluoroethylene, expanded polytetrafluoroethylene, or silicone. It may be more preferable that the plastic chosen for forming the space filling element be silicone. Other gel type plastics, or similar materials known by those skilled in the art may also be used.

There may be disposed on the inner face 16 an adhesive 18. The adhesive 18 may be attached to the inner face 16 in a manner which does not readily detach from the inner face 16 in the conditions typically found in a human oral cavity. The adhesive 18 may also adhere to the gums when disposed against them. It may be preferable that such adherence be a pressure-sensitive adherence, wherein the adhering properties of the adhesive 18 are caused by the application of pressure when brought in contact against an upper or lower gum. It may also be preferable that a moisture-sensitive adhesive 18 be used, wherein the moisture-sensitive adhering properties of the adhesive 18 are activated by being brought in contact with a moist environment, such as the saliva present on the upper or lower gums in the oral cavity. It may additionally be preferable that the adherence between the adhesive 18 and the gums be readily reversable by the wearer without excessive pain or discomfort to allow for selective removal of the prosthetic from the oral cavity. Such removal of the prosthetic device may be accomplished by, for example but without limitation, pulling off the prosthetic or weakening or dissolving the adhesive 18 with a suitable solvent.

A suitable adhesive 18 may be, for example but without limitation, acrylates, methylacrylates, diacrylates, cyanoacrylates, abietic acids, gum arabic, poly(methylvinylether-co-maleic acid) or polyvinyl acetates. It may be more preferable that the adhesive 18 chosen be poly(methylvinylether-co-maleic acid). Other adhesives or alternatives to adhesives known by those skilled in the art may also be used. The adhesive 18 may be pre-applied by the manufacturer. The adhesive 18 may alternatively be applied by the wearer prior to wearing the prosthetic device 10.

A preferred embodiment may include a removable release liner 20 disposed over the adhesive 18 such that the adhesive 18 is sandwiched between the inner face 16 and the release liner 20. The release liner 20 may serve to protect the integrity, cleanliness, and adhesive properties of the adhesive 18 prior to use of the prosthetic device 10 by the wearer. In such embodiments, immediately prior to use of the prosthetic device 10, the wearer may remove the release liner 20 in order to expose the adhesive 18. It may be preferable in such embodiments that the release liner 20 be discarded after being removed. In other such embodiments, it may alternatively be preferable that the release liner 20 be replaceable over the adhesive 18 if the prosthetic device 10 is intended to be reusable. Although the preferred embodiment includes removable release liner 20, it is also contemplated that other embodiments do not include a release liner 20.

It may be preferable to include a flavoring or breath freshening element with the space filling element 12. Such flavoring or breath freshening elements may include, for example but without limitation, coatings or impregnations of flavored or breath freshening substances. In some embodiments, the adhesive 18 or release liner 20 may be flavored or have breath freshening properties.

A prosthetic device 10 according to another embodiment of the present invention may have one or more segment lines 22. The segment lines 22 may be used to adjust the size of the prosthetic device 10 so as to better fit against the gums of each individual wearer.

Referring now to FIG. 2, it may be preferable that the segment lines 22 be disposed at constant and repeating increments so as to allow a wearer to adjust the size of a prosthetic device 10 according to a previously known or measured configuration. For example, in an embodiment in which the prosthetic device 10 may be intended to be discarded after a single use, a wearer may have previously determined their preferred dimensions, and may then rapidly configure the prosthetic device 10 to their preferred dimension by removing material beyond one or more segment lines 22. In certain embodiments, the segment lines 22 may be dotted lines along which a wearer may cut with cutting instruments such as scissors or other bladed tools. In other embodiments, the segment lines 22 may comprise a series of perforations along which a wearer may rapidly tear, removing the necessity for a separate tool. Alternatively, the segment lines 22 may comprise a nick or cut, allowing the wearer to rapidly tear the material of the prosthetic device 10 beginning along that nick or cut. The removed segment 22 may then be discarded by the wearer prior to use.

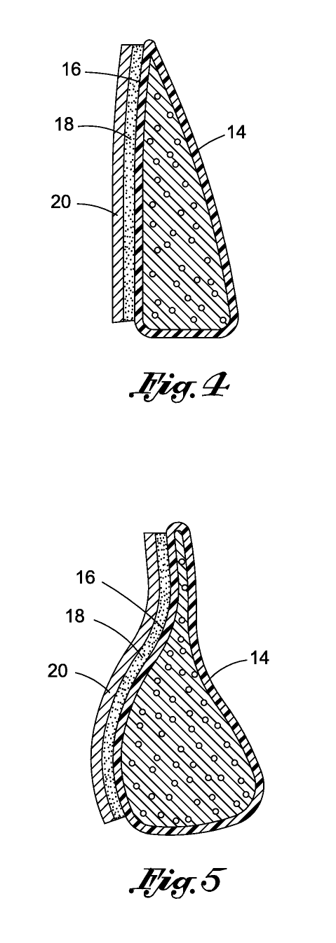

Referring now to FIG. 3, a cross section in a transverse plane of an embodiment of the prosthetic device 10 is shown. It may be seen that such an embodiment may have an arcuate, generally crescent-shaped cross-section in which the outer face 14 is convex and the inner face 16 is concave.

Referring now to FIG. 4, a cross section in a transverse plane of an alternative embodiment of the prosthetic device 10 is shown. It may be seen that such an alternative embodiment may have a generally wedge-shaped cross section in which the outer face 14 and the inner face 16 are generally planar, and the distance between the inner and outer faces 14 and 16 generally increases along the cross section. In such an embodiment, it may be preferable to dispose the wider end of the space filling element 12 nearer to the gingival margin. When worn on the upper gums, the wider end may be oriented downward as shown in the drawing. When worn on the lower gums, the prosthetic may be inverted with respect to the orientation in the drawing, with the wider end facing upward. Such a wedge-shaped cross section may result in a more pronounced augmentation of the lips.

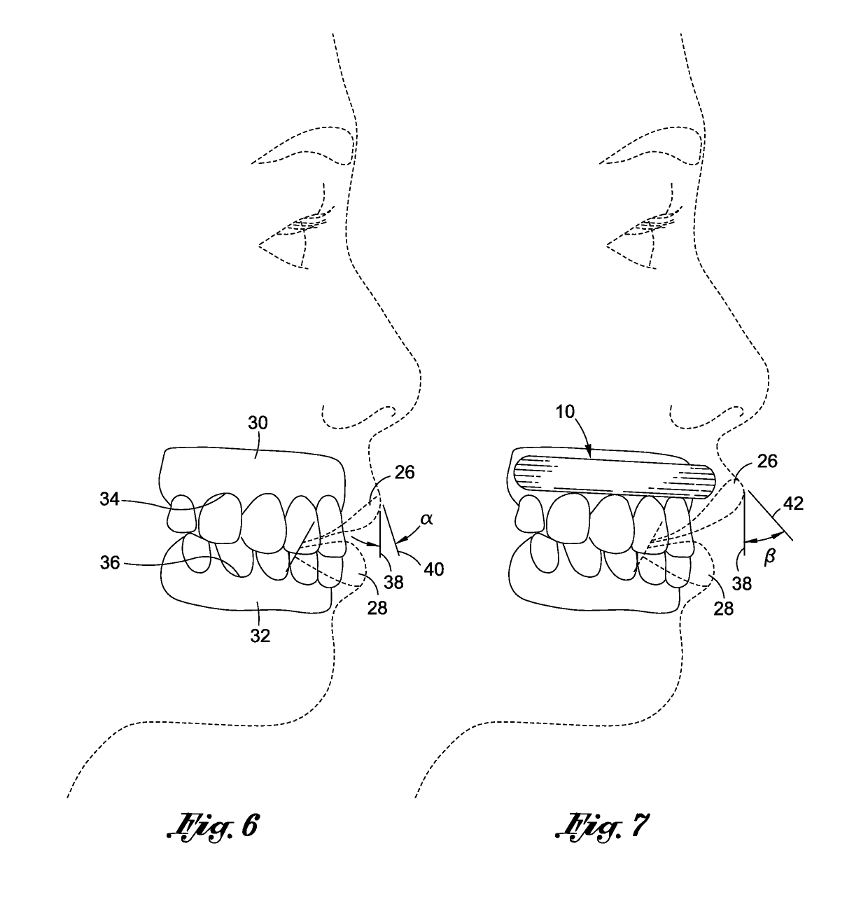

Referring now to FIG. 5, a cross section in a transverse plane of a further alternative embodiment of the prosthetic device 10 is shown. It may be seen that in such a further alternative embodiment, the cross section may be generally teardrop shaped, in which the outer face 14 and the inner face 16 curve outward towards the wider end of the space filling element 12. Such an embodiment may provide an even more pronounced lip augmentation than in either of the previously discussed embodiments of FIG. 3 or 4. However, it may also be seen that the prosthetic device 10 may be configured to be any size and dimension suitable for its intended purpose and use.

Referring now to FIG. 6, a profile illustration of a face and oral cavity prior to wearing of the prosthetic is shown. It may be seen that in an oral cavity prior to wearing of the prosthetic, the upper lip 26 may rest against the upper gingiva 30, and the lower lip 28 may rest against the lower gingiva 32. Also visible in the illustration are the upper and lower gingival margins 34 and 36, which are the points on the gingiva defining the visible border between the gums and the teeth. As may be seen, without the prosthetic device, an angle a may be seen to be formed between a vertical axis 38 and an unagumented axis 40 defined by the slope of the upper lip 26. To increase the apparent fullness of the lips, it may be desired to increase that angle.

Referring now to FIG. 7, a profile illustration of a face and oral cavity is shown wherein a prosthetic device 10 according to an embodiment of the present invention is worn. It may be seen that the inner face 16 of the space filling element 12 may be adhered by the adhesive 18 to the upper gingiva 30 so as to cause the upper lip 26 to more distinctly protrude outward, as the upper lip 26 no longer rests against the upper gingiva 30, but rather may rest against the outer face 14 of the space filling element 10. It is readily understood that the when the space filling element 12 is placed against the lower gingiva 32, the lower lip 28 may be caused to more distinctly protrude as well in similar fashion. In such a way, the fullness of the wearer's lips may be augmented without ready detection by onlookers that the wearer's lip fullness is being aided by a prosthetic. As may be seen, with the presence of the prosthetic device 10, the apparent increase in fullness of the lips is illustrated by an angle β which may be formed between a vertical axis 38 and an augmented axis 42 defined by the slope of the upper lip 26 when the prosthetic device 10 is worn. Said angle β may be larger than angle a of the previous FIG. 6 due to the placement of the space filling element 12 between the upper gingiva 30 and the upper lip 26.

It may thus be seen that an aspect of the present invention may be that when the adhesive 18 is adhered to the upper or lower gingiva 30 or 32, the prosthetic device 10 does not protrude substantially beyond the gingival margins 34 or 36 of the gingiva against which the prosthetic is placed. In a preferred embodiment, the prosthetic device 10 does not protrude beyond the gingival margins 34 or 36. However, it may also be seen that the characteristics of the gingiva and gingival margin of each individual wearer of the prosthetic device 10 may vary, and as such the prosthetic device 10 may occasionally protrude beyond the gingival margins 34 or 36 for some wearers, such as those why may have thinner or receding gums. The general consideration is that the prosthetic device 10 should not readily be exposed to onlookers when the wearer's teeth are normally exposed, for example, while the wearer is speaking or smiling, so as to preserve the illusion that the wearer has naturally full lips. As such, some amount of protrusion beyond the gingival margins 34 or 36 may be acceptable, without diverging from the scope and spirit of the invention.

The above description is given by way of example, and not limitation. Given the above disclosure, one skilled in the art could devise variations that are within the scope and spirit of the invention disclosed herein, including various ways of sizing or configuring the space filling element, or adhering the adhesive 18 to the inner face 16 or the upper or lower gingiva 30 or 32. Further, the various features of the embodiments disclosed herein can be used alone, or in varying combinations with each other and are not intended to be limited to the specific combination described herein. Thus, the scope of the claims is not to be limited by the illustrated embodiments.

What is claimed is:

1. A prosthetic device placeable in an oral cavity having a gingiva and a gingival margin, for temporarily achieving the appearance of fuller lips, said prosthetic device comprising:
a space filling element comprising opposing inner and outer faces and sized and configured to be complimentary to an upper or lower gingiva;
said space filling element being sized and configured to be selectable placeable adjacent to one of an upper or lower gingiva and substantially nonprotrusive beyond the gingival margin of the chosen gingiva; and
an adhesive on the inner face of the space filling element, said adhesive being configured to temporarily adhere to an upper or lower gingiva;
wherein when the space filling element is adjacent to an upper or lower gingiva, a lip is pushed outward by the outer face.

2. The prosthetic device of claim 1, wherein the space filling element is formed of a gel-type plastic.

3. The prosthetic device of claim 2, wherein the gel-type plastic is chosen from the group: polyethelene, polyurethane, polypropylene, polystyrene, polyvinyl chloride, polytetrafluoroethylene, expanded polytetrafluoroethylene or silicone.

4. The prosthetic device of claim 3, wherein the gel-type plastic is silocone.

5. The prosthetic device of claim 1, wherein the adhesive is chosen from the group: acrylate, methylacrylate, diacrylate, cyanoacrylate, abietic acid, gum arabic, poly(methylvinylether-co-maleic acid) or polyvinyl acetate.

6. The prosthetic device of claim 5, wherein the adhesive is poly(methylvinylether-co-maleic acid).

7. The prosthetic device of claim 1, wherein the adhesive is a pressure-sensitive adhesive.

8. The prosthetic device of claim 1, wherein the adhesive is a moisture-sensitive adhesive.

9. The prosthetic device of claim 1, further comprising a removable release liner removably adhered to the adhesive.

10. The prosthetic device of claim 1, wherein the space filling element is configured with one or more marked segment lines for selectively forming the size and dimension of the space filling element to fit an upper or lower gingiva.

11. The prosthetic device of claim 1, further comprising a flavoring element coupled to the space filling element.

12. The prosthetic device of claim 1, further comprising a breath-freshening element coupled to the space filling element.

13. The prosthetic device of claim 1, wherein the space filling element is sized and configured to mitigate interference with a wearer's ability to speak, eat, or drink in a normal manner when the adhesive is temporarily adhered to an upper or lower gingiva.

14. A method for placing a prosthetic in an oral cavity having a gingiva and a gingival margin, for temporarily achieving the appearance of fuller lips, the method having the steps of:
providing a space filling element having:
opposing inner and outer faces sized and configured to be selectable placeable adjacent to an upper or lower gingiva and substantially nonprotrusive beyond the gingival margin of the chosen gingiva; and
an adhesive adhered to the inner face of the space filling element; and
removably adhering the adhesive to an upper or lower gingiva.

15. The method of claim 14, wherein the provided space filling element further comprises a release liner removably adhered to the adhesive.

16. The method of claim 15, further comprising the step of removing the release liner removably adhered to the adhesive prior to the adhering step.

17. A method of fitting a prosthetic device for temporarily achieving the appearance of fuller lips for placement on a gingiva having a gingival margin, the method comprising the steps of:
providing a space filling element having:
opposing inner and outer faces sized and configured to be selectable placeable adjacent to an upper or lower gingiva and substantially nonprotrusive beyond the gingival margin of the chosen gingiva; and
an adhesive adhered to the inner face of the space filling element; and
adjusting the prosthetic device to a size suitable for placement on an upper or lower gingiva;

18. The method of claim 17, wherein the provided space filling element further comprises one or more marked segment lines.

19. The method of claim 18, wherein the adjusting step is accomplished by cutting the space filling element along one or more of the one or more marked segment lines.

20. The method of claim 18 wherein the adjusting step is accomplished by tearing the space filling element along one or more of the one or more marked segment lines.
